(19) United States
(12) Patent Application Publication
(10) Pub. No.: US 2020/0337683 A1
(43) Pub. Date: Oct. 29, 2020

(54) SAMPLING CATHETER WITH ARTICULATING TIP

(71) Applicant: THE UNITED STATES GOVERNMENT AS REPRESENTED BY THE DEPARTMENT OF VETERANS AFFAIRS, Washington, DC (US)

(72) Inventors: Gianfranco Umberto Meduri, Germantown, TN (US); Garrett Grindle, Pittsburgh, PA (US)

(21) Appl. No.: 16/960,999

(22) PCT Filed: Jan. 10, 2019
(86) PCT No.: PCT/US19/12977

§ 371 Date: Jul. 9, 2020

Related U.S. Application Data
(60) Provisional application No. 62/616,117, filed on Jan. 11, 2018.

(51) Int. Cl. A61B 10/00; A61M 25/01; A61B 1/05; A61B 1/06; A61B 1/005; A61B 1/015

(52) U.S. Cl. CPC A61B 10/0045; A61M 25/0136; A61M 25/0147; A61M 25/0138; A61B 1/015; A61B 1/0661; A61B 1/0052; A61B 1/0057; A61B 1/05

(57) ABSTRACT

A catheter assembly uses articulating wires which extend from a traction mechanism mounted on a control handle to a distal end of the catheter to steer the catheter as it traverses internal cavities of a patient. A camera and a light may be mounted on the catheter for use with an imaging monitor for visual guidance of the catheter. A luer fitting and a valve mounted on the control handle are used to control the flow of fluid into and out of the catheter lumen.

### CROSS REFERENCE TO RELATED APPLICATIONS

This application is based upon and claims priority to U.S. Provisional Application No. 62/616,117, filed Jan. 11, 2018 and hereby incorporated by reference.

### FIELD OF THE INVENTION

This invention concerns catheters for retrieving samples from anatomical areas of the body.

### BACKGROUND

Diagnosis of and screening for diseases such as pneumonia are advantageously accomplished through the collection of uncontaminated secretions from anatomical areas of the body. Bronchoalveolar lavage (BAL) is a technique to sample a large anatomical area of the lung to increase diagnostic sensitivity. BAL through a protected catheter (distal ejectable biodegradable plug) provides increased specificity by minimizing contamination. Collection of secretions directly from the lower respiratory tract improves diagnostic yield for pneumonia. Methods for collection of lower respiratory tract secretions include bronchoscopic (under visualization) and non-bronchoscopic (blind) bronchoalveolar lavage using catheters such as those disclosed in U.S. Pat. No. 5,289,560 (Method of Protected Bronchial Sampling Using a Translaryngoscopic Catheter) and U.S. Pat. No. 5,474,542, (Catheter Having Imperforate Protective Barrier and Method for Making and Using the Same), these patents being hereby incorporated by reference.

One disadvantage of the non-bronchoscopic techniques is that the placement of the sampling catheter is a blind procedure. It is difficult to direct the catheter through the bronchial tree and to ascertain if the sample has been collected from the desired area of the lung (right vs left; upper vs. lower). In essence the catheter is advanced blindly until resistance is met and the operator cannot verify that the necessary seal is achieved before proceeding with BAL. Sealing is important to maximize return of BAL fluid. Although the catheters are flexible they are substantially unguided as they traverse deeper into the bronchial tree. There is clearly an opportunity to improve the performance of catheters for the collection of secretions in the diagnosis of and screening for diseases.

### SUMMARY

The invention concerns a catheter assembly for instillation and aspiration of fluids including therapeutic agents. In an example embodiment the catheter assembly comprises a catheter defining at least a first lumen. The catheter has a distal end and a proximal end. A control handle is mounted on the proximal end of the catheter. A traction mechanism is movably mounted on the control handle. A first articulation wire is attached to and extends from the distal end of the catheter through the first lumen to the traction mechanism. Motion of the traction mechanism pulls the first articulation wire and causes the distal end to bend in response.

In an example embodiment the traction mechanism comprises a first button slidably mounted on the control handle. A first crank arm is mounted on the control handle for rotation about an axis. A first link is pivotably attached between the first button and the first crank arm on a first side of the axis. The first articulation wire is attached to the first crank arm on a second side of the axis opposite to the first side of the axis.

In an example embodiment the traction mechanism further comprises a second button slidably mounted on the control handle and a second crank arm mounted on the control handle for rotation about an axis. A second link is pivotably attached between the second button and the second crank arm on a first side of the axis of the second crank arm. A second articulation wire is attached to and extends from the distal end of the catheter through the first lumen. The second articulation wire is attached to the second crank arm on a second side of the axis of the second crank arm opposite to the first side of the axis of the second crank arm.

In an example embodiment the traction mechanism comprises a crank arm mounted on the control handle for rotation about an axis. A lever extends from the crank arm. The first articulation wire is attached to the crank arm on a first side of the axis. A second articulation wire is attached to and extends from the distal end of the catheter through the first lumen. The second articulation wire is attached to the crank arm on a second side of the axis opposite to the first side.

In an example embodiment the traction mechanism comprises an electrical motor having an output shaft. A controller controls rotation of the output shaft. A differential gear train is coupled to the output shaft. The differential gear train comprises first and second shafts rotatable in opposite directions by the electrical motor. The first articulation wire is attached to the first shaft. A second articulation wire is attached to and extends from the distal end of the catheter through the first lumen. The second articulation wire is attached to the second shaft. Rotation of the output shaft in a first direction places tension on the first articulation wire and slack on the second articulation wire, thereby causing the distal end of the catheter to bend in a first direction, and rotation of the output shaft in an opposite direction places tension on the second articulation wire and slack on the first articulation wire, thereby causing the distal end of the catheter to bend in a second direction different from the first direction.

A further example embodiment comprises a plurality of openings extending through the catheter over a region of the distal end. The openings increase the bending flexibility of the distal end of the catheter. By way of example, the openings comprise a plurality of slots arranged on opposite sides of the catheter. The slots extend in a circumferential direction about the first lumen. In another example embodiment, the distal end of the catheter comprises a first segment hingedly attached to the catheter, a terminal segment, and a plurality of intermediate segments hingedly attached to one another and extending between the first segment and the terminal segment. In this example each intermediate segment comprises a first and a second hinge at opposite ends thereof. Each first hinge has a first pivot axis, and each second hinge has a second pivot axis oriented transversely to the first pivot axis.

In an example embodiment the catheter defines a second lumen positioned within the first lumen. The second lumen extends from the distal to the proximal end of the catheter. An example embodiment may further comprise a luer fitting in fluid communication with the second lumen. A valve controls fluid flow through the second lumen. In an example embodiment the luer fitting is positioned at the proximal end of the catheter. By way of example the valve may be integral with the luer fitting.

An example embodiment may further comprise a removable plug positioned within the second lumen at the distal end of the catheter. By way of further example, a light source may be positioned within the first lumen at the distal end of the catheter, and a camera may be positioned within the first lumen at the distal end of the catheter. A plurality of electrical conductors extend through the first lumen for suppling power to the light source and the camera and for transmitting optical signals from the camera.

In an example embodiment the catheter may be disconnectable from the control handle. In one example embodiment, the catheter comprises a unitary catheter having the proximal end removably connected to a luer fitting mounted on the control handle. At least the first articulation wire extends through the first lumen and exits the first lumen short of the proximal end for attachment to the traction mechanism. In another example embodiment the catheter comprises a first length permanently affixed to the control handle. The first length includes the proximal end of the catheter. A second length is removably connected to the first length. The second length includes the distal end of the catheter. Further by way of example, a coupling attaches the first length to the second length. A fitting removably attaches the first articulation wire to the traction mechanism.

### BRIEF DESCRIPTION OF THE DRAWINGS

FIG. 1 is an isometric view of an example catheter assembly according to the invention;

FIG. 2 is an isometric, partial sectional view of an example catheter assembly;

FIG. 3 is an isometric, partial sectional view of an example catheter assembly;

FIG. 4 is an isometric, partial sectional view of an example catheter assembly;

FIG. 5 is an isometric view of a portion of an example catheter assembly;

FIGS. 6 and 7 are side views of a portion of an example catheter assembly;

FIG. 8 is an isometric view of a portion of an example catheter assembly;

FIG. 9 is an isometric view of a portion of an example catheter assembly;

FIG. 10 is an isometric, partial sectional view of a portion of an example catheter;

FIG. 11 is an isometric, partial sectional view of a portion of an example catheter;

FIG. 11A is an isometric, partial sectional view of a portion of an example catheter;

FIG. 12 is an isometric view of an example catheter assembly in use; and

FIGS. 13 and 14 are detailed views of a portion of an example catheter assembly in use.

### DETAILED DESCRIPTION

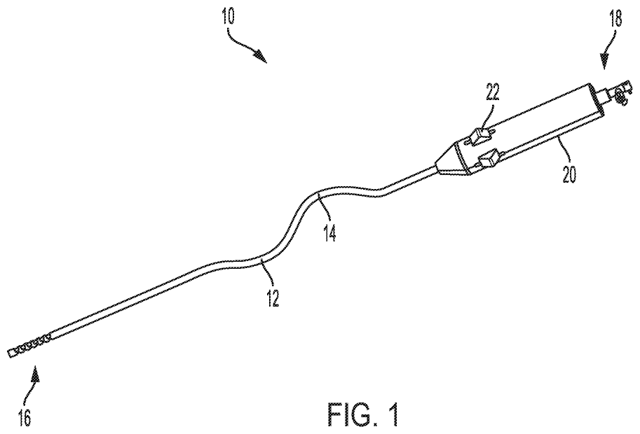

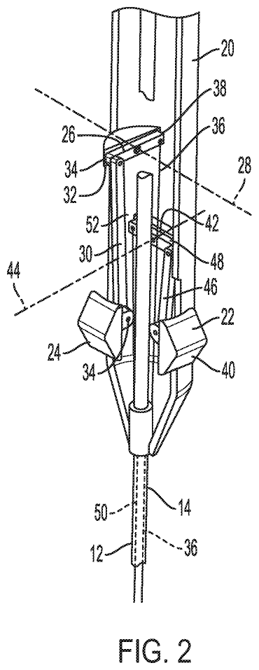

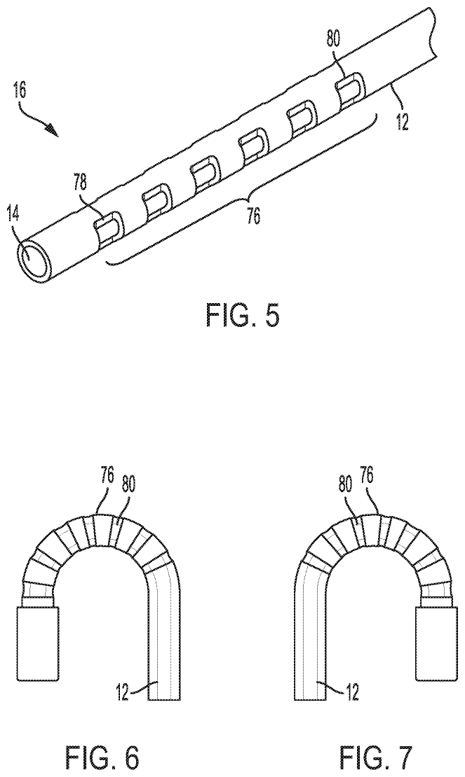

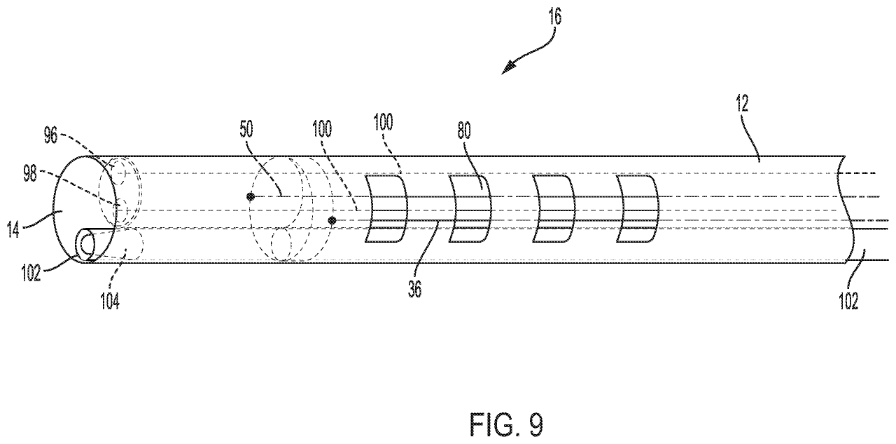

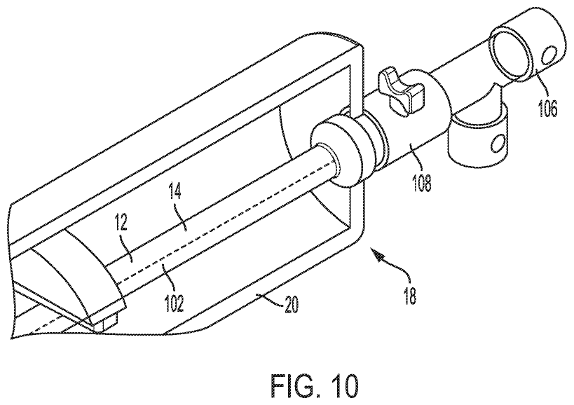

FIG. 1 shows an example embodiment of a catheter assembly 10 according to the invention. Assembly 10 comprises a catheter 12 defining a first lumen 14 (see also FIGS. 5 and 9). Catheter 12 is flexible and formed of any one of a number of medical grade materials such as silicone, polyurethane, polyethylene, polyvinylchloride, polytetrafluoroethylene, nylon and rubber latex. Catheter 12 has a distal end 16 and an oppositely disposed proximal end 18. A control handle 20 is mounted on catheter 12 at its proximal end. Control handle 20 allows manual manipulation of the assembly 10 and comprises a traction mechanism 22 movably mounted thereon for articulation of the distal end 16 of catheter 12. An example traction mechanism 22 is shown in FIG. 2 and comprises a first button 24 slidably mounted on the control handle 20. A first crank arm 26 is mounted on the control handle 20, the crank arm 26 being rotatable about a crank arm axis 28. A first link 30 attaches the button 24 to the crank arm 26 on a first side 32 of the axis 28. Link 30 is connected to the button 24 and crank arm 26 via pivot joints 34 to permit sliding motion of the button to rotate the crank arm 26 about its axis 28. A first articulation wire 36 is attached to crank arm 26 on a second side 38 of axis 28 opposite to the first side 32. Wire 36 may be made of braided stainless steel and extends from the crank arm 26 through the lumen 14 to the distal end 16 of catheter 12 (see FIG. 9). Motion of the button 24 which puts the articulation wire 36 in tension will bend the catheter 12 at its distal end (as described below) to steer the catheter as it traverses body cavities, trachea, bronchi, ducts, or vessels during medical procedures.

As further shown in FIG. 2, the traction mechanism 22 of the example catheter assembly 10 may comprise a second button 40 also slidably mounted on the control handle 20. A second crank arm 42 is mounted on the control handle 20, the crank arm 42 being rotatable about a second crank arm axis 44. A second link 46 attaches the button 40 to the crank arm 42 on a first side 48 of the axis 44. Like link 30, link 46 is connected to the button 40 and crank arm 42 via pivot joints 34 to permit sliding motion of the button to rotate the crank arm 42 about its axis 44. A second articulation wire 50 is attached to crank arm 42 on a second side 52 of axis 44 opposite to the first side 48. Like wire 36, wire 50 may be made of braided stainless steel and extends from the crank arm 42 through the lumen 14 to the distal end 16 of catheter 12 (see FIG. 9). Two buttons 24 and 40 operating respective crank arms 26 and 42 attached to respective articulation wires 36 and 50 attached to opposite sides of catheter 12 cooperate to apply tension to one or the other articulation wire 36 or 50 as necessary to bend the catheter 12 at its distal end in different directions to steer the catheter during medical procedures.

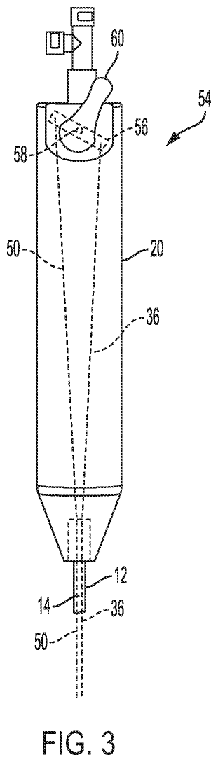

FIG. 3 shows another example traction mechanism 54 which comprises a single crank arm 56 mounted on control handle 20 for rotation about an axis 58. A lever 60 extends from the crank arm 56 to permit easy manual rotation of the crank arm 56. In this example embodiment the first articulation wire 36 is attached to crank arm 56 on one side of its axis 58, and the second articulation wire 50 is attached to the crank arm 56 on the other side of its axis 58. As the articulation wires extend through the lumen 14 and are attached to opposite sides of the catheter 12 at the distal end 16, pivoting motion of the crank arm 56 about its axis 58 using the lever 60 will place tension on one of the articulation wires while simultaneously allowing slack on the other to bend the distal end 16 of the catheter 12 and steer it during a procedure.

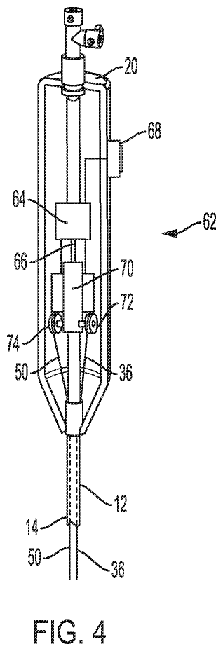

FIG. 4 shows another example traction mechanism 62 mounted within control handle 20. Traction mechanism 62 comprises an electrical motor 64 having an output shaft 66. A controller 68, such as a two pole switch, controls the direction of motion of the output shaft 66. Motor 64 may be run by a battery (not shown) or electrical service. A differential gear train 70 is coupled to the output shaft 66. Gear train 70 has first and second shafts 72 and 74 which rotate in opposite directions upon rotation of the output shaft 66 in either direction. The first articulation wire 36 is attached to the first shaft 72 and the second articulation wire 50 is attached to the second shaft 74. Rotation of the output shaft 66 of the motor 64 in a first direction rotates the first shaft 72 so as to place tension on the first articulation wire 36 while simultaneously rotating the second shaft 74 in the opposite direction to allow slack on the second articulation wire 50. As the articulation wires extend through the lumen 14 and are attached to opposite sides of the catheter 12 at the distal end 16 (see FIG. 9), this rotation of output shaft 66 acting through the differential gear train, will bend the distal end 16 of the catheter 12 to steer it. Reversal of the motor 64 (via the controller 68) drives the output shaft 66 in the opposite direction to place tension on the second articulation wire 50 while simultaneously allowing slack on the first articulation wire 36 and bend the distal end 16 of the catheter 12 in the opposite direction.

While the catheter 12 is flexible along it length, as shown in FIG. 5, it is advantageous to increase its bending flexibility over a region 76 of the distal end 16. Increasing the bending flexibility of the distal region 76 permits the catheter 12 to bend to smaller radii of curvature with less force than if the catheter were not as flexible. FIG. 5 shows an example of a distal end region 76 having increased bending flexibility caused by a plurality of openings 78 extending through the catheter 12. In this example embodiment the openings comprise slots 80 arranged on opposite sides of the catheter 12. The slots 80 extend in a circumferential direction about the lumen 14 and allow side to side bending as shown in FIGS. 6 and 7. It is advantageous to attach the articulation wires 36 and 50 to the distal region 76 by weaving them through the slots 80 as shown in FIG. 9, and thereby apply tension force where the catheter is most flexible.

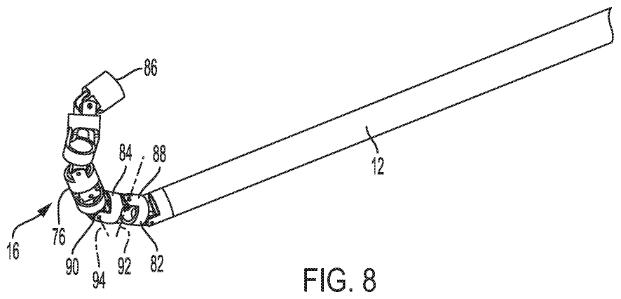

FIG. 8 shows another embodiment of a catheter distal end 16 having increased bending flexibility. In this example the distal end region 76 comprises a first segment 82 hingedly attached to the catheter 12. A plurality of intermediate segments 84 are hingedly attached to one another, to the first segment 82 and to a terminal segment 86. The articulation wires (not shown) are attached to opposite sides of the terminal segment 86 to effect its bending. Each intermediate segment 84 has respective first and second hinges 88 and 90 at opposite ends, and each hinge has a respective hinge axis 92 and 94. It is advantageous if the first and second hinge axes 92 and 94 of each segment are oriented transversely to one another to permit bending about multiple axes when multiple articulation wires are used.

FIG. 9 shows the distal end 16 of an example catheter 12 comprising a light source 96 and a camera 98 positioned within the lumen 14. In this example catheter assembly, a plurality of electrical conductors 100 extend through the lumen 14 for supplying power to the light source and camera, and to transmit optical signals from the camera to a viewing device (not shown). Light source 96 may be, for example, a light emitting diode. Such a catheter assembly would be useful for visual exploration of body cavities, trachea, bronchi, ducts, and vessels, to perform for example, bronchoalveolar lavage (BAL). This example catheter is adapted for BAL or other sampling procedures by the addition of a second lumen 102 defined by the catheter 12 and positioned within the first lumen 14. Lumen 102 extends from the distal end 16 to the proximal end 18 of catheter 12. A removable plug 104 is positioned within the lumen 102 at the distal end 16. The plug 104 maintains the sterility of the lumen 102 as it traverses the patient before arriving at the target area. Once the distal end 16 is at the target the plug 104 is ejected and sampling procedures or local therapeutic interventions can be performed as described below. This is a simple and rapid technique to monitor and diagnose pneumonia and other lung diseases that can be applied in a variety of clinical settings, including Emergency Room Department, hospital ward (bedside), and intensive care unit. In addition to physicians, a wide range of medical personnel (physician assistants, nurses, nurse practitioners, respiratory therapists, etc.) can make use of the proposed catheter. Improved accuracy in reaching the desired anatomical area while avoiding contamination of the inner catheter lumen provides superior diagnostic and therapeutic results. Moreover, the catheter can be used for diagnostic and therapeutic purposes in anatomical compartments outside the lung like the pleural space, peritoneal cavity, gastrointestinal tract, urinary bladder, and gallbladder. For selected use, the articulation wires allow for visualization of catheter's placement under ultrasound guidance.

As shown in FIG. 10 a luer fitting 106 is attached to the proximal end 18 of catheter 12 and in fluid communication with the second lumen 102. A valve 108, for example, a ball valve, is used to control the flow of fluid through the second lumen 102. In this example the valve 108 is integrally formed with the luer fitting 106. Luer fitting 106 allows fluid to be introduced into and recovered from lumen 102 during the lavage procedure as described below. Similarly, therapeutic or diagnostic agents can be locally instilled.

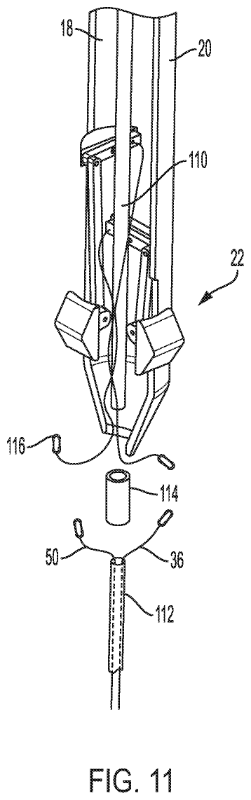

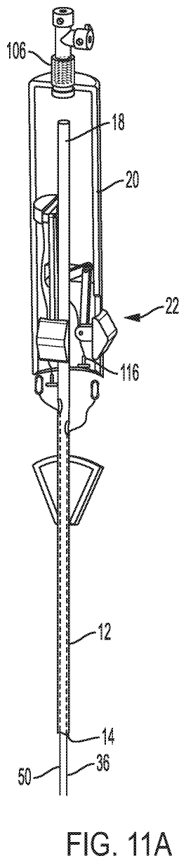

It is advantageous to permit a portion of the catheter 12 to be detached from the assembly 10. This detachability permits portions of the assembly to be discarded after use, while portions of the assembly, such as the control handle 20, may be reused. In the example embodiment shown in FIG. 11, a first length 110 of catheter 12 comprising the proximal end 18 is permanently affixed to the control handle 20, and a second length 112, comprising the majority of the catheter 12 and including the distal end 16 (not shown) is removably attached to the first length 110. Attachment may be via a coupling 114, which could use friction to secure the lengths 110 and 112 to one another. Other types of couplings including compression couplings and bayonet couplings are also feasible. Additional fittings 116, such as spring biased clips, may be used to removably attach the articulation wires 36 and 50 to the traction mechanism 22. FIG. 11A shows an alternate embodiment of the catheter assembly having a unitary (i.e., single piece) disposable catheter 12 whose proximal end 18 (shown disconnected) is removably attached to the luer fitting 106. In this embodiment both the first and second articulation wires 36 and 50 extend through the lumen 14 and exit the catheter short of its proximal end 18. The control handle 20 may be partially disassembled to connect the articulation wires 36 and 50 to the traction mechanism 22. Luer fitting 106 is also removably attached to handle 20, for example, using screw threads. Any portion of the catheter assembly exposed to a patient's fluids must be removable from handle 20 and disposable.

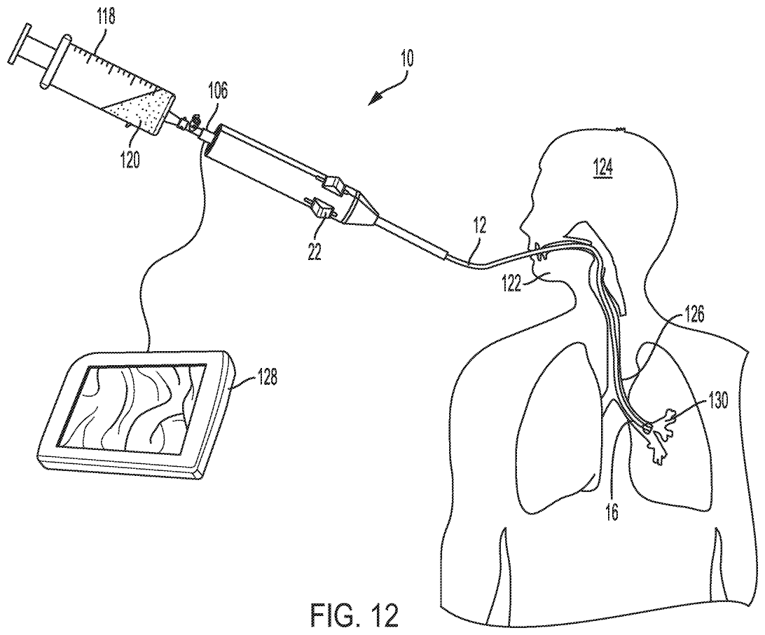

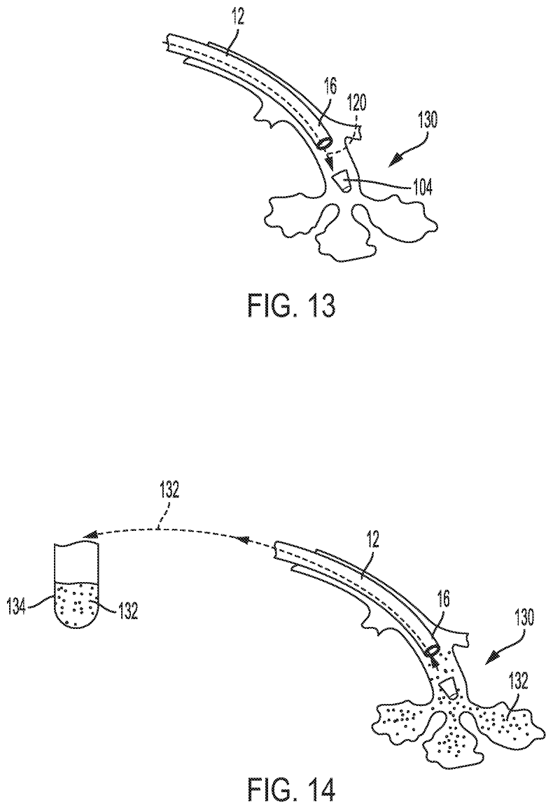

FIGS. 12-14 illustrate use of an example catheter assembly 10 for a bronchoalveolar lavage procedure. As shown in FIG. 12 a syringe 118 having BAL fluid 120 is engaged with the luer fitting 106 for filling the second (operating) lumen 102 (see FIGS. 9 and 10). The amount of fluid injected and aspirated is approximately 150 cc (five 30 cc syringes), but can vary. As shown in FIG. 9, the operating lumen 102 is sealed with a plug 104 to prevent contamination of the operating lumen as it traverses the tracheo-bronchial tree or other part of anatomy through which the catheter is passed. Catheter 12 is then passed through the oral cavity 122 of the patient 124 and into the trachea-bronchial tree 126. For patients on mechanical ventilation, the catheter 12 may be advanced through an endotracheal tube (not shown). The operator guides the catheter 12 using the images captured by the camera 98 and displayed continuously in real time on an imaging monitor 128. Using the traction mechanism 22, the distal end 16 of catheter 12 is bent to steer the catheter to the desired position within the lower respiratory tract 130 (see FIG. 13). Once the distal end 16 of catheter 12 is in the desired location and a seal is obtained within a bronchus, BAL is initiated (see FIGS. 13 and 14) resulting in the ejection of the protective plug 104. Plug 104 is bio-degradable, and is ejected from the lumen 102 when the first portion of BAL fluid 120 is advanced from syringe 18 after the distal end 16 of catheter 12 is positioned as desired. Through visualization on monitor 128 (using the camera 98, illuminated by the light source 96), the operator can maximize accurate sampling and return of secretions 132 from the lower respiratory tract 130 (see FIG. 12). Once an adequate BAL sample 134 is retrieved, the operator removes the catheter under direct visualization. The BAL sample 134 is then sent to the laboratory for diagnostic analysis.

The catheter assembly 10 according to the invention is versatile because it enables embodiments of varying sophistication, for example, the embodiment having two articulation wires, a power articulation control and an imaging system including camera, light and monitor. In an alternate, less expensive embodiment, the device may have a single articulation wire, a manual articulation control and no imaging system. Furthermore, the catheter assembly 10 according to the invention may be made partially or totally disposable, thereby mitigating the risk of infection and contamination.

What is claimed is:

1. A catheter assembly for sampling fluids, said catheter assembly comprising:
a catheter defining at least a first lumen, said catheter having a distal end and a proximal end;
a control handle mounted on said proximal end of said catheter;
a traction mechanism movably mounted on said control handle;
a first articulation wire attached to and extending from said distal end of said catheter through said first lumen to said traction mechanism, motion of said traction mechanism pulling said first articulation wire and causing said distal end to bend in response.

2. The catheter assembly according to claim 1, wherein said traction mechanism comprises:
a first button slidably mounted on said control handle;
a first crank arm mounted on said control handle for rotation about an axis;
a first link pivotably attached between said first button and said first crank arm on a first side of said axis; and
said first articulation wire being attached to said first crank arm on a second side of said axis opposite to said first side of said axis.

3. The catheter assembly according to claim 2, wherein said traction mechanism further comprises:
a second button slidably mounted on said control handle;
a second crank arm mounted on said control handle for rotation about an axis;
a second link pivotably attached between said second button and said second crank arm on a first side of said axis of said second crank arm; and
a second articulation wire attached to and extending from said distal end of said catheter through said first lumen, said second articulation wire being attached to said second crank arm on a second side of said axis of said second crank arm opposite to said first side of said axis of said second crank arm.

4. The catheter assembly according to claim 1, wherein said traction mechanism comprises:
a crank arm mounted on said control handle for rotation about an axis;
a lever extending from said crank arm; wherein
said first articulation wire is attached to said crank arm on a first side of said axis.

5. The catheter assembly according to claim 4, further comprising a second articulation wire attached to and extending from said distal end of said catheter through said first lumen, said second articulation wire being attached to said crank arm on a second side of said axis opposite to said first side.

6. The catheter assembly according to claim 1, wherein said traction mechanism comprises:
an electrical motor having an output shaft;
a controller for controlling rotation of said output shaft;
a differential gear train coupled to said output shaft, said differential gear train comprising first and second shafts rotatable in opposite directions by said electrical motor, said first articulation wire being attached to said first shaft; and
a second articulation wire attached to and extending from said distal end of said catheter through said first lumen is attached to said second shaft, wherein
rotation of said output shaft in a first direction places tension on said first articulation wire and slack on said second articulation wire, thereby causing said distal end of said catheter to bend in a first direction, and rotation of said output shaft in an opposite direction places tension on said second articulation wire and slack on said first articulation wire, thereby causing said distal end of said catheter to bend in a second direction different from said first direction.

7. The catheter assembly according to claim 1, further comprising a plurality of openings extending through said catheter over a region of said distal end, said openings increasing the bending flexibility of said distal end of said catheter.

8. The catheter assembly according to claim 7, wherein said openings comprise a plurality of slots arranged on opposite sides of said catheter, said slots extending in a circumferential direction about said first lumen.

9. The catheter assembly according to claim 1, wherein said distal end of said catheter comprises:
a first segment hingedly attached to said catheter;
a terminal segment;
a plurality of intermediate segments hingedly attached to one another and extending between said first segment and said terminal segment.

10. The catheter assembly according to claim 9, wherein each intermediate segment comprises a first and a second hinge at opposite ends thereof, each said first hinge having a first pivot axis, each said second hinge having a second pivot axis oriented transversely to said first pivot axis.

11. The catheter assembly according to claim 1, wherein said catheter defines a second lumen positioned within said first lumen, said second lumen extending from said distal to said proximal end of said catheter.

12. The catheter assembly according to claim 11, further comprising:
a luer fitting in fluid communication with said second lumen;
a valve controlling fluid flow through said second lumen.

13. The catheter assembly according to claim 12, wherein said luer fitting is positioned at said proximal end of said catheter.

14. The catheter assembly according to claim 13, wherein said valve is integral with said luer fitting.

15. The catheter assembly according to claim 11, further comprising a removable plug positioned within said second lumen at said distal end of said catheter.

16. The catheter assembly according to claim 1, further comprising:
a light source positioned within said first lumen at said distal end of said catheter;
a camera positioned within said first lumen at said distal end of said catheter.

17. The catheter assembly according to claim 16, further comprising a plurality of electrical conductors extending through said first lumen for suppling power to said light source and said camera and for transmitting optical signals from said camera.

18. The catheter assembly according to claim 1, wherein said catheter is disconnectable from said control handle.

19. The catheter assembly according to claim 18, wherein said catheter comprises a unitary catheter having said proximal end removably connected to a luer fitting mounted on said control handle, at least said first articulation wire extending through said first lumen and exiting said first lumen short of said proximal end for attachment to said traction mechanism

20. The catheter assembly according to claim 18, wherein said catheter comprises:
a first length permanently affixed to said control handle, said first length including said proximal end of said catheter;
a second length removably connected to said first length, said second length including said distal end of said catheter.

21. The catheter assembly according to claim 20, further comprising a coupling for attaching said first length to said second length.

22. The catheter assembly according to claim 18, further comprising a fitting for removably attaching said first articulation wire to said traction mechanism.
